(19) United States
(12) Patent Application Publication
(10) Pub. No.: US 2026/0117256 A1
(43) Pub. Date: Apr. 30, 2026

(54) CELL ENGINEERING TARGET CELLS IN A MIXTURE COMPRISING THE TARGET CELLS AND NON-TARGET CELLS

(71) Applicant: IMEC VZW, Leuven (BE)

(72) Inventors: Tim Stakenborg, Heverlee (BE); Rocco Stirparo, Leuven (BE); Peter Peumans, Leuven (BE)

(21) Appl. No.: 19/373,242

(22) Filed: Oct. 29, 2025

(30) Foreign Application Priority Data
Oct. 31, 2024 (EP) 24210256.4

(51) Int. Cl. C12N 15/87; C12M 1/34; C12M 3/00; C12M 3/06

(52) U.S. Cl. CPC C12N 15/87; C12M 23/16; C12M 23/44; C12M 41/36

(57) ABSTRACT

The present disclosure provides a system for cell engineering target cells in a mixture that includes the target cells and non-target cells, the system including: i) a cell monitoring component for tracking and classifying individual cells in the mixture, wherein classifying a cell comprises determining whether the cell is a target cell; ii) an engineering component for selectively modifying individual cells at one or more engineering sites, wherein the target cells and the non-target cells are randomly distributed in the engineering component; and iii) a control component for controlling the engineering component so as to selectively modify only target cells tracked to be at one of the engineering sites.

### CROSS-REFERENCE TO RELATED APPLICATION

The present application is a non-provisional patent application claiming priority to European Patent Application No. EP 24210256.4, filed Oct. 31, 2024, the contents of which are hereby incorporated by reference.

### TECHNICAL FIELD

The present disclosure relates to the field of cell engineering, and more specifically to selectively modifying individual target cells within a mixture of target and non-target cells.

### BACKGROUND

In the field of cellular biology and medical research, the ability to modify (e.g. genetically) cells at the single-cell level is crucial e.g. for advancements in therapy and for furthering the understanding of cellular functions. Traditional approaches to modifying target cells in a mixture of cells typically involve sorting out the target cells, followed by transduction or transfection of the target cells. These approaches thus typically require the physical separation of target cells from a heterogeneous mixture, which can be inefficient and yield worse results. Additionally, such approaches may handle cells in batches rather than on an individual basis, limiting control of cell modification tasks.

Additionally, sorting is generally done using label-based methods, such as fluorescence-activated cell sorting (FACS) or magnetic-activated cell sorting (MACS). These techniques, while effective for certain applications, require the cells to be labelled (e.g. with fluorescent markers for FACS or magnetic beads for MACS). This labelling process not only adds additional steps (e.g. additional sample preparation and wash steps to remove unbound labels or reagents) to the procedure—making it time-consuming, complex and more at risk of cell loss or damage—but also introduces foreign materials that can alter cell behaviour/function and/or cell viability. These issues may be exacerbated when dealing with rare cell types or when sample sizes are limited.

Cell engineering can be applied to cell therapy. The immune system plays a pivotal role in defending the body against infections and diseases. It possesses the ability to adapt and respond to various pathogens and internal threats, including viruses, bacteria, cancerous cells, fibrosis and aging. However, there are instances where the immune system fails to make the right tool to fight back (e.g. for HIV), is too slow and needs to be prepared upfront (e.g. with vaccines), or struggles when the threat is an alteration of the body's own cells (e.g. in the case of cancer or aging).

To bolster the immune system's capacity to fight diseases, various strategies have been developed to prime the immune system to fight a disease of interest. For example, vaccination prepares the immune system in advance to combat infections, such as influenza or COVID-19. Another approach includes cell therapies, such as chimeric antigen receptor T (CAR-T) cell therapies, which can include modifying the receptors of T-cells to specifically bind to a particular target antigen on certain cells (e.g. cancer cells). While CAR-T therapies have demonstrated promising results, especially in treating previously untreatable cancers, the production of these therapies is challenging.

Previously available manufacturing processes for cell therapies are complex and time-consuming, often requiring several weeks to produce a single dose. This can involve multiple steps such as cell extraction, selection, modification, and expansion, each necessitating specialized equipment, facilities and personnel. Extensive quality control tests at each stage can increase the overall cost and duration of therapy production. The logistics involved further complicate accessibility, as most manufacturing facilities are centralized, necessitating the transportation of biological materials over long distances, potentially compromising their viability and efficacy.

Cell engineering is also extensively used in lab/research settings; e.g. in gene expression studies, knock-down/out studies, protein production, stem cell research, high-throughput screening, miRNA studies, etc.

### SUMMARY

Some embodiments of the present disclosure includes an apparatus for selectively modifying individual target cells in a mixture comprising the target cells and non-target cells. This outcome may be accomplished by systems, modules, cartridges, and/or methods as described herein.

Some embodiments of the present disclosure are capable of selectively modifying target cells, such that only targeted cells are modified. Some embodiments of the present disclosure may be capable of modifying target cells without first isolating them from a mixture comprising both target and non-target cells. Some embodiments of the present disclosure facilitate the classification and selection of cells without the need for external labels such as magnetic beads or fluorescent tags, simplifying the process and reducing the risk of contamination.

Some embodiments of the present disclosure preform cell monitoring and engineering in a continuous flow, and even at a relatively high cell-throughput. Such continuous flow can be a constant flow or a (non-constant) modulated flow, e.g. by surface modification as described in EP24205936.8, which is incorporated herein by reference. Some embodiments of the present disclosure include making decisions regarding the cells to be modified at the single-cell level, allowing for precise and targeted cell engineering.

Some embodiments of the present disclosure allows multiple individual cells to be classified and tracked in parallel, significantly increasing efficiency. Some embodiments of the present disclosure perform cell monitoring in real-time over a relatively wide area.

Some embodiments of the present disclosure operate as a closed loop system, maintaining the integrity and sterility of samples throughout the process. This not only enhances safety but also reduces the amount and degree of associated quality control performed.

Some embodiments of the present disclosure can be used for both therapeutic (e.g. cell therapy) and non-therapeutic (e.g. research) applications.

Some embodiments of the present disclosure can be performed in a decentralized manner (e.g. directly at the point of care or in a laboratory), reducing or eliminating the need for transportation of samples and/or long travel times for the patient and/or researcher.

Exemplary aspects of the present disclosure are set out in the accompanying independent and dependent claims. Features from the dependent claims may be combined with features of the independent claims and with features of other dependent claims as appropriate and not merely as explicitly set out in the claims.

The above and other characteristics, features, and benefits of the subject matter of the present disclosure will become apparent from the following detailed description, taken in conjunction with the accompanying drawings, which illustrate, by way of example, the principles of the present disclosure. This description is given for the sake of example only, without limiting the scope of the subject matter contemplated herein. The reference figures quoted below refer to the attached drawings.

### BRIEF DESCRIPTION OF DRAWINGS

FIG. 1 schematically depicts a system and a cell engineering module, in according to an example embodiment.

FIG. 2 schematically depicts a system that incorporates a cell engineering module, according to an example embodiment.

FIG. 3 schematically depicts a cell engineering module in the form of a cartridge and a device for receiving the cartridge, according to an example embodiment.

FIG. 4 schematically depicts a top view of a potential cell engineering module, imaging unit, and control component, according to an example embodiment.

FIG. 5 schematically depicts a side view of the cell engineering module and control component of FIG. 4, according to an example embodiment.

FIG. 6 schematically depicts a top view of a further potential cell engineering module, imaging unit, and control component, according to an example embodiment.

FIG. 7 schematically depicts a side view of the cell engineering module and control component of FIG. 6 according to an example embodiment.

FIG. 8 is a block diagram of a vein-to-vein system, according to an example embodiment.

FIG. 9 is a block diagram of a lab system, according to an example embodiment.

In the different figures, the same reference signs refer to the same or analogous elements.

### DETAILED DESCRIPTION

The subject matter of the present disclosure is described with respect to particular embodiments and with reference to certain drawings but the subject matter contemplated herein is not limited thereto. The drawings described are schematic and are non-limiting. In the drawings, the size of some of the elements may be exaggerated and not drawn to scale for illustrative purposes. The dimensions and the relative dimensions do not necessarily correspond to specific actual reductions to practice of the present disclosure.

The terms first, second, third and the like in the description and in the claims, are used for distinguishing between similar elements and not necessarily for describing a sequence, either temporally, spatially, in ranking, or in any other manner. It is to be understood that the terms so used are interchangeable under appropriate circumstances and that the embodiments described herein are capable of operation in other sequences than described or illustrated herein.

The terms top, bottom, over, under, and the like in the description and the claims are used for descriptive purposes and not necessarily for describing relative positions. It is to be understood that the terms so used are interchangeable with their antonyms under appropriate circumstances and that the embodiments described herein are capable of operation in other orientations than described or illustrated herein.

The term “comprising,” as used in the claims, should not be interpreted as being restricted to the means listed thereafter; it does not exclude other elements or steps. It is thus to be interpreted as specifying the presence of the stated features, integers, steps or components as referred to, and does not preclude the presence or addition of one or more other features, integers, steps, or components or groups thereof. The term “comprising” therefore covers the situation where only the stated features are present and the situation where these features and one or more other features are present. Thus, the scope of the expression “a device comprising means A and B” should not be interpreted as being limited to devices consisting only of components A and B. It means that with respect to the subject matter of the present disclosure, the only relevant components of the device are A and B.

The term “coupled”, also used in the claims, should not be interpreted as being restricted to direct connections only. The terms “coupled” and “connected”, along with their derivatives, may be used. These terms are not intended as synonyms for each other. Thus, the scope of the expression “a device A coupled to a device B” should not be limited to devices or systems wherein an output of device A is directly connected to an input of device B. It means that there exists a path between an output of A and an input of B which may be a path including other devices or means. “Coupled” may mean that two or more elements are either in direct physical or electrical contact, or that two or more elements are not in direct contact with each other but still co-operate or interact with each other.

Reference throughout this specification to “one embodiment” or “an embodiment” means that a particular feature, structure or characteristic described in connection with the embodiment is included in at least one embodiment of the present disclosure. Thus, appearances of the phrases “in one embodiment” or “in an embodiment” in various places throughout this specification are not necessarily all referring to the same embodiment, but may. Furthermore, the particular features, structures, or characteristics depicted herein may be combined in any suitable manner as would be apparent from this disclosure.

In the description of exemplary embodiments of the present disclosure, various features are sometimes grouped together in a single embodiment, figure, or description thereof for the purpose of streamlining the disclosure and aiding in the understanding of one or more of the various aspects thereof. This method of disclosure, however, is not to be interpreted as reflecting an intention that the subject matter of the present disclosure requires more features than are expressly recited, e.g., in each claim. Rather, aspects of the subject matter contemplated herein may lie in less than all features of a single foregoing disclosed embodiment. Thus, the claims following the detailed description are hereby expressly incorporated into this detailed description, with each claim standing on its own as a separate embodiment of the present disclosure.

While some embodiments described herein include some but not other features included in other embodiments, combinations of features of different embodiments are meant to be within the scope of the present disclosure, and form different embodiments, as would be understood by those in the art. For example, in the following claims, any of the claimed embodiments can be used in any combination.

In the description provided herein, numerous specific details are set forth. However, it is understood that embodiments of the present disclosure may be practised without these specific details. In other instances, well-known methods, structures and techniques have not been shown in detail in order not to obscure an understanding of this description.

The following terms are provided solely to aid in the understanding of the present disclosure.

As used herein, and unless otherwise specified, the term ‘cell engineering’ refers to the purposeful process of modifying a (living) cell. This may generally include inserting, deleting, and/or altering a component in the cell. In specific instances, the component may be a genetic sequence, such as a nucleic acid sequence (e.g. deoxyribonucleic acid, DNA; or ribonucleic acid, RNA). This may for example be achieved by genetic transformation. Within the present disclosure, the cell which is modified may be a prokaryote or a eukaryote, though more typically a eukaryote.

As used herein, and unless otherwise specified, the term ‘genetic transformation’ refers to the genetic modification/alteration of a cell resulting from the direct uptake and incorporation of genetic material—for example exogenous/foreign genetic material—from its surroundings through the cell membrane(s). Specific example of genetic transformation include transduction—by which the genetic material is introduced into the cell by a virus or viral vector—or transfection—by which the genetic material is introduced into the cell by non-viral means. Transfection can for example be achieved through opening of the cell (herein also referred to as ‘poration’) by physical means (e.g. electroporation, photoporation, sonoporation, cell squeezing or magnetofection) or (bio)chemical means (e.g. calcium phosphate, lipofection, etc.).

As used herein, and unless otherwise specified, the term ‘target cell’ refers to a cell of interest which is to undergo cell engineering. By contrast, a ‘non-target cell’ refers to a cell which isn't a target for the cell engineering (and is not intended to undergo the modification). Within the present disclosure, cell engineering is generally performed in a mixture comprising both target cells and non-target cells.

As used herein, and unless otherwise specified, the term ‘cell monitoring’ refers to a process of tracking and classifying individual cells in a mixture. Here, ‘classifying a cell’ comprises determining whether the cell is a target cell. Cell monitoring may for example be performed within a cell monitoring zone, using an imaging unit. A ‘cell monitoring component’ is component capable of performing cell monitoring. Such a cell monitoring component may thus comprise a cell monitoring zone and an imaging unit. A specific type of cell monitoring is ‘label-free cell monitoring,’ wherein the cell monitoring takes place without the use of labels/tags/markers (such as fluorescent dyes, antibodies, magnetic beads, or any other exogenous substances by which the cells may be labelled).

As used herein, and unless otherwise specified, the term ‘computational imaging’ refers to using computation as part of the image formation process or to reveal data-driven insights using algorithms from captured data. In other words, computational imaging relies on algorithms and models to construct, modify or interpret a captured image (i.e. using optics) or even to form images from measurements. Such processes may rely on a significant amount of computing, but often facilitate reconstruction/deduction of information that traditional cameras cannot capture and/or that was not present in an original image. An example is holographic imaging which uses interference of light waves to capture a hologram and subsequently computationally process the hologram. Computational imaging can involve reconstructing an actual image or processing raw data to deduce imaging information therefrom without actually reconstructing an image as such. For example in holographic imaging, the measurement may be the capture of an interference pattern (i.e. a hologram; e.g. using a digital image sensor), from which a 3D detailed image (revealing the shape, size, location, etc. of the objects yielding the interference pattern) is computationally reconstructed. Alternatively, no image as such may be reconstructed, but instead the relevant information (e.g. size, location, etc.) on the objects (e.g. cells) may be derived directly from the measured hologram. Examples of other (label-free) computational imaging methods include high-resolution ptychographic imaging, 3D ptychographic imaging, optical diffraction tomography microscopy, and label-free structured illumination microscopy. Compared to traditional imaging (i.e. using optics to capture an image as such, without computational enhancement), computational imaging creates or enhances the imaging to a level that may not be achievable relying on optics alone.

As used herein, and unless otherwise specified, the term ‘engineering component’ refers to a component capable of performing cell engineering on a cell. Within the present disclosure, the engineering component may be capable of selectively (e.g. under control by the control component) modifying-e.g. by genetic transformation (cf. supra)-individual cells at one or more engineering sites.

As used herein, and unless otherwise specified, the term ‘control component’ refers to a component capable of controlling another component or system. For example, a control component may be capable of controlling the engineering component, such as controlling an activation thereof. The latter may include one or more of controlling the triggering of an electric, light, sound or magnetic pulse (e.g. to open the cell), or controlling the release of one or more (bio)chemicals (e.g. a (bio)chemical for opening the cell and/or a nucleic acid to be inserted into the cell). To this end, the control component may comprise a processor or other processing means configured for performing its control function. The control component may enact its control function based on one or more inputs, and thus the control component may comprise a processor or other similar processing means (the same or a different one) to process these inputs.

As used herein, and unless otherwise specified, the term ‘cell movement modifying surface’ refers to a surface capable of interacting distinctly with different types of cells, such that a trajectory of some cell types is distinctly altered as compared to other cell types. The cell movement modifying surface may for example have a higher affinity (e.g. a physical, chemical and/or biochemical affinity) for one type of cell as compared to another type of cell.

As used herein, and unless otherwise specified, the term ‘cell type’ (or ‘cell category’) refers to any cell categorization as may be useful in a relevant context. For instance, one cell type could be all cells which have a certain expression level of a marker, or of a combination of multiple markers. Another cell type could be all cells having a certain physical morphology (e.g. shape and/or size).

As used herein, and unless otherwise specified, the term ‘biological mixture’ refers to a mixture comprising biological entities (e.g. cells, organelles, nucleic acids, proteins, carbohydrates, lipids, etc.). Such a mixture may be of biological origin (e.g. a bodily fluid) or artificial origin (e.g. cells in a buffer).

As used herein, and unless otherwise specified, the term ‘apheresis’ refers to a process by which a particular substance or component is removed from blood. Such a substance or component may further be referred to as the ‘fraction of interest’ and may include target cells; while the rest—i.e. the blood from which the fraction of interest has been separated—may be referred to as the ‘residual fraction’. The residual fraction may be returned to the body/patient. In some instances, also the fraction of interest is—after cell engineering—returned to the body/patient. The latter may be done together with or separate from the residual fraction. Specific types of apheresis include plasmapheresis (removing blood plasma), erythrocytapheresis (removing red blood cells), plateletpheresis (also referred to as ‘thrombapheresis’ or ‘thrombocytapheresis’, removing blood platelets), leukapheresis (removing leukocytes/white blood cells), lymphapheresis (removing lymphocytes) and stem cell harvesting (removing hematopoietic stem cells).

As used herein, and unless otherwise specified, the term ‘vein-to-vein system’ refers to a system the operation of which starts with drawing blood from a patient (‘extraction’; e.g. from a vein) and ends with transferring modified target cells back into a patient (‘reinsertion’; the patent may be the same or a different patient). In other words, the blood is temporarily withdrawn, not permanently removed. In some embodiments, this may be performed in a ‘closed loop’, wherein the extracted blood remains in the system from extraction to reinsertion.

In a first aspect, the present disclosure relates to a system for cell engineering target cells in a mixture comprising the target cells and non-target cells, comprising: i) a cell monitoring component for tracking and classifying individual cells in the mixture, wherein classifying a cell comprises determining whether the cell is a target cell; ii) an engineering component for selectively modifying individual cells at one or more engineering sites, wherein the target cells and the non-target cells are randomly distributed in the engineering component; and iii) a control component for controlling the engineering component so as to selectively modify only target cells tracked to be at one of the engineering sites.

In some embodiments, the mixture may be a biological mixture, for example a bodily fluid or a fraction thereof. The bodily fluid may for instance be blood or lymph. Although non-therapeutic uses are anticipated, the subject matter of the present disclosure may in be useful in cell therapy performed directly on bodily fluids or fractions thereof. For example, for performing procedures such as CAR-T immunotherapy.

In some embodiments, the target cells may be immune cells or stem cells, for example immune cells. In some embodiments, the immune cells may be peripheral blood mononuclear cells, for example lymphocytes, for example T cells or NK cells. Engineering these cell types can have therapeutic—but also non-therapeutic (e.g. research)—applications.

In some embodiments, the cell monitoring component may comprise an imaging unit, for example a computational microscopy module, for example a lens-free imaging unit. Compared to imaging using lenses, lens-free imaging can allow for a considerably larger field of view (FoV; e.g. 15 mm² or more), thereby enabling longer (i.e. more spatially extended) tracking and monitoring. This longer tracking and monitoring can provide higher accuracy for cell identification, as well as for the selection of when to control the engineering component to selectively modify the target cells at one of the engineering sites. (Computational microscopy) lens-free imaging thus facilitates high-throughput (supporting relatively high flow rate) cell monitoring simultaneously over a wide area, thereby allowing efficient classification and tracking of the individual cells over the full cell monitoring zone using a single imaging unit (as opposed to e.g. needing to stitch together information/images from multiple imaging units). Alternatively, a plurality of the imaging units can be used together—e.g. including stitching and integrating together the image of each—such that the imaging zone is increased, beyond, e.g., traditional imaging. This can further increase the yield of the system. This can facilitate label-free cell monitoring, thereby addressing some of the problems associated with cell labelling (cf. Background).

In some embodiments, the imaging unit may comprise an illumination source and an image sensor. In some embodiments, the illumination source (or ‘light source’) may be configured for illuminating objects (e.g. cells) in the cell monitoring component (e.g. in the cell monitoring zone). For example, the light source may emit coherent light. Depending on the application, the emitted light may be monochromatic or polychromatic (as e.g. described in EP2879004A1 and/or LI, Yuqian, et al. Accurate label-free 3-part leukocyte recognition with single cell lens-free imaging flow cytometry. Computers in biology and medicine, 2018, 96:147-156.; which are incorporated herein by reference). An interference pattern may thereby be formed by interference between light being scattered by the illuminated objects and non-scattered light from the illumination source. In embodiments, the image sensor (e.g. a digital image sensor) may be configured for capturing the interference pattern; e.g. as a digital hologram. In embodiments using computational imaging, the system may comprise a processor configured for receiving the captured interference pattern and processing it into an image. In embodiments, the system may comprise a processor configured for receiving one or more captured interference patterns as such or one or more of the images, and deriving therefrom information (e.g. shape, size, transparency, scattering, speed, direction and/or path) about the objects (e.g. cells) illuminated by the illumination source. In some embodiments, the aforementioned processors may be a single processor or may be multiple separate processors. In some embodiments, the single or multiple processors may be (independently) comprised in the image sensor as such, the imaging unit, the control component, or another external processor unit. Such an imaging unit may not include a lens, potentially making it more compact (lenses and related optics make a microscope bulky), robust (lens alignments are not needed), and affordable. Moreover, it may—compared to traditional microscopes—have a comparable resolution but a much larger field of view (and thus a much bigger imaged area). In some examples, the computational imaging may nevertheless comprise a lens.

Other types of cell monitoring—label-free or otherwise—are also within the scope of the present disclosure. For example, the cell monitoring component may comprise a more traditional image sensor, e.g., comprising a lens. In alternative or complementary embodiments, the cell monitoring component may comprise a plurality of image sensors. Multiple image sensors can allow imaging a larger area, even where the individual image sensors have relatively low field of view; keeping track of individual cells can be more challenging as they move from one imaging window to the next. When using multiple image sensors, each image sensor may have its own dedicated illumination source, or two or more image sensors may share an illumination source.

In some embodiments, the cell monitoring component may comprise a fluidic channel. In some embodiments, the mixture may flow through the cell monitoring component via the fluidic channel. In some embodiments, the fluidic channel may be a microfluidic channel. In some embodiments, the system may be a microfluidic system (e.g. a microfluidic device). The use of compact (micro)fluidic channels and system/device architectures keeps the surface area with the which the mixture may interact low, thereby reducing the risk of contamination of the mixture.

In some embodiments, the cell monitoring component may comprise a cell movement modifying surface. In some embodiments, the fluidic channel may comprise the cell movement modifying surface. The cell movement modifying surface can facilitate (label-free) cell classification based on cell-surface interactions.

Cells may comprise cell membrane molecules which can act as cell identification molecules, such as antigens. Certain molecules, e.g. antibodies, may bind specifically to such cell membrane molecules. For example, antigen CD4 (cluster of differentiation 4) is a glycoprotein which binds to anti-CD4 antibodies. Accordingly, cells that bind to anti-CD4 antibodies may form one cell category. Such cells may be detected based on their modified movements (e.g. modified speed, direction, and/or path) when they travel through a zone with a cell movement modifying (CMM) surface coated by anti-CD4 antibodies. More specifically, the cells with antigen CD4 may undergo several bind-and-release events with anti-CD4 antibodies as they travel through the zone. Analogously, cells that bind to anti-CD8 (cluster of differentiation 8) antibodies may form another cell category. Such cells may be detected based on their modified movements when they travel through a zone with a cell movement modifying surface coated by anti-CD8 antibodies. Binding and releasing the cell may temporarily halt the cell along its travel. This yields an altered motion in the form of a change in speed, direction and/or path, which is detectable and allows the cell to be classified.

In some embodiments, the cell movement modifying surface may have an affinity for a particular type of cell over another type of cell, e.g. for a particular cell identification molecule over another cell identification molecule. For example, the cell movement modifying surface may comprise—or may be coated with—antibodies, aptamers, lectins and/or any other molecule that specifically binds to a cell. In embodiments, the cell modifying surface may bind the cell for a finite amount of time. In embodiments, the cell movement modifying surface may further be as described in EP23219766.5, which is incorporated herein by reference.

In some embodiments, the cell monitoring component (e.g. the imaging unit) may be configured to detect an altered movement of a cell in the cell monitoring component (e.g. the cell monitoring zone); such as one or more bind-and-release events, a halt of motion, a change of speed, a change of direction, or a change of path. In some embodiments, the cell monitoring component (e.g. the imaging unit) may be configured to detect a threshold number (e.g. at least 2, for example at least 5, for example at least 10) of the aforementioned altered movements. The detected altered movement then facilitates classification of the cell.

In some embodiments, the fluidic channel may comprise one or more structures having the cell movement modifying surface. Such structures may have any shape; for example, the structures may be an array of pillars. Such structures can provide a high surface area for the cell-surface interactions. The use of such structures is optional and the cell movement modifying surface may also be provided on a (flat) surface of the fluidic channel.

In some embodiments, the fluidic channel may comprise multiple subzones having distinct cell movement modifying surfaces. Using multiple distinct movement modifying surfaces allows additional degrees of selection, thereby raising the accuracy of the cell classification, and thereby the accuracy of the cell modification. For example, while the interactions of two similar cell types (e.g. having comparable physical and (bio)chemical properties) with any single movement modifying surface may be too similar to confidently tell apart, their differential interactions with a plurality of different movement modifying surfaces may provide a clearer picture and allow them to be distinguished. Such configurations can also allow for a plurality of different types of target cells (e.g. in a complex mixture) to be distinguished.

In some embodiments, determining whether the cell is a target cell may be based on a determination of morphological parameters (e.g. shape and/or size), optical parameters (e.g. transparency and/or scattering), and/or kinetic parameters (e.g. speed direction, and/or path) of the cell. Such properties allow between different types (e.g. categories) of cells to be distinguished.

In some embodiments, the engineering sites may be arranged with respect to the flow of the mixture such that every cell is likely to pass at least one engineering site prior to exiting the engineering module. This can increase the likelihood that the cell modification can be applied to every target cell. In some embodiments, not every cell passes at least one engineering site. For some applications, it is not necessary for all target cells to be modified; e.g. the procedure may be effective (although possibly less so) if only a certain percentage (less than 100%) of the target cells are actually modified.

Tracking an individual cell may involve monitoring the position of the cell over time. In some embodiments, tracking of individual cells in the mixture (e.g. during and/or after classifying them) may be performed (at least) up to the engineering sites in the engineering component. Keeping track of classified cells up to the engineering component can help to ensure that only cells that are identified to be target cells are modified in the engineering component. In some embodiments, tracking of individual cells in the mixture (e.g. during and/or after classifying them) may be performed up to a point prior to the engineering sites. This may be possible if the movement of the tracked cells can be predicted from that point onwards. Such a configuration can improve the efficiency of the system (e.g. by taking up less resources overall per tracked cell), but can also be a source of errors if the actual cell movement deviates significantly from the prediction. In some embodiments, only the target cells may continue to be tracked up to the engineering sites, while in other embodiments all cells may continue to be tracked up to the engineering sites. While tracking only the target cells may be sufficient to allow selectively modifying only the target cells, also tracking the non-target cells can facilitate additional checking that no non-target cells are at risk of being modified (e.g. because they are too close to a target cell).

Where tracking continues at least up to the engineering sites, the engineering component may be at least partially (e.g. completely) contained within the cell monitoring component. For example, the cell monitoring component may comprise a cell monitoring zone (cf. supra and infra) and the engineering component may be at least partially (e.g. completely) within the cell monitoring zone. The mixture may flow through the cell monitoring component (zone) and into the engineering component, and the cell classification may be performed in the cell monitoring component (zone) upstream relative to the engineering component. Where tracking is performed up to a point prior to the engineering sites, the engineering component may be coupled (e.g. connected) to the cell monitoring component. Accordingly, the mixture may still flow through the cell monitoring component (zone) and—optionally via a further zone/component—into the engineering component, and the cell classification may be performed in the cell monitoring component (zone) upstream relative to the engineering component.

In some embodiments, selectively modifying individual cells may include selectively inserting and/or modifying a nucleic acid in the individual cells. The modification of the cells may thus include a genetic transformation. In some embodiments, selectively modifying the individual cells may include selectively transfecting the individual cells. Transfection can enable efficient delivery of nucleic acids into the cells. In some embodiments, transfecting may comprise electroporating, photoporating, cell squeezing, magnetofecting, chemical transfecting, or biochemical transfecting. This versatile selection of techniques can be effective at opening (porating) the cell for modification (e.g. for genetic transformation).

In some embodiments, a composition of the mixture may be maintained between classifying the individual cells and selectively modifying the individual cells (e.g. not including the selective modifying, as at least some reagents may be added to the mixture at that point). In some embodiments, nothing may be removed from the mixture between classifying the individual cells and selectively modifying the individual cells. In the latter embodiments, something may be added to the mixture (e.g. reagent, solvent, etc.). The selective modification is thus beneficially not depending on a separate cell sorting step (e.g. separating the target cells from the non-target cells), but on individually selecting for modification only those cells classified as target cells and tracked to be at one of the engineering sites.

In some embodiments, any feature of any embodiment of the first aspect may independently be as correspondingly described for any embodiment of any of the other aspects.

While embodiments of the present disclosure have above been described as a facet of a larger system, the subject matter contemplated herein may also be configured as a module for integration into a larger system. Accordingly, in a second aspect, the present disclosure relates to a cell engineering module for cell engineering target cells in a mixture comprising the target cells and non-target cells, comprising: i) a cell monitoring zone for allowing tracking and classifying individual cells in the mixture, wherein classifying a cell comprises determining whether the cell is a target cell; and ii) an engineering component in the cell monitoring zone for selectively modifying individual cells at one or more engineering sites, wherein the target cells and the non-target cells are randomly distributed in the engineering component; wherein the engineering component is adapted for—in operation—being controlled so as to selectively modify only target cells tracked to be at one of the engineering sites.

In some embodiments, the cell monitoring zone may be present within a fluidic channel. In some embodiments, the mixture may flow through the cell monitoring zone via the fluidic channel. In some embodiments, the fluidic channel may be a microfluidic channel. In some embodiments, the cell engineering module may be a microfluidic module. The use of compact (micro)fluidic channels and system/module architectures keeps the surface area with the which the mixture may interact low, thereby reducing the risk of contamination of the mixture.

In some embodiments, the cell monitoring zone may comprise a cell movement modifying surface. In some embodiments, the cell monitoring zone may comprise at least one transparent surface for imaging cells in the cell monitoring zone. The cell movement modifying surface may be, function and operate is described for the first aspect.

In some embodiments, the cell monitoring zone may comprise at least one transparent surface—for example at least two opposing transparent surfaces—for imaging cells in the cell monitoring zone. When the imaging unit is external to the cell engineering module, one or more transparent (e.g. substantially transparent to the wavelengths used for the cell imaging by the imaging unit) surfaces allow tracking and classification in the cell monitoring component using the imaging unit.

In some embodiments, the engineering component may be adapted for interfacing with a control component. For example, the control component may be coupled—e.g. wired or wirelessly—to an input of the engineering component. Interfacing with a control component facilitates automated selective cell modification in the module. This allows control over the selective modification of the engineering component by the control component (based on the classification and tracking in the cell monitoring component).

In some embodiments, any feature of any embodiment of the second aspect may independently be as correspondingly described for any embodiment of any of the other aspects.

In a third aspect, the subject matter of the present disclosure relates to a cartridge comprising the cell engineering module according to any embodiment of the second aspect.

A cartridge format provides a convenient platform for the cell engineering module, allowing those parts of the system which came into contact with the mixture to be easily removed and disposed of after use. Conversely, the parts which do not come into contact with the mixture (e.g. the imaging unit and control component) can be integrated in a system adapted for receiving the cartridge (cf. infra), so that they can be easily reused (e.g. do not need to be disposed with the cartridge), thereby reducing the operation and hardware costs of the system.

In some embodiments, any feature of any embodiment of the third aspect may independently be as correspondingly described for any embodiment of any of the other aspects.

In a fourth aspect, the subject matter of the present disclosure relates to a system comprising a cell engineering module as defined in any embodiment of the second aspect, or adapted to receive a cartridge as defined in any embodiment of the third aspect.

In some embodiments, the system being adapted to receive the cartridge may include the system including an interface adapted to receive (e.g. ‘slotting in’) the cartridge.

In some embodiments, the system may further include a control component for controlling the engineering component so as to selectively modify only target cells tracked to be at one of the engineering sites.

In some embodiments, the system may further comprise an imaging unit, for example a computational microscopy module, for example a lens-free imaging unit.

In some embodiments, the system may further comprise—before the cell engineering module—an apheresis module for separating blood into a fraction of interest comprising the target cells, and a residual fraction. The apheresis module facilitates separating the fraction comprising the target cells from fractions not comprising the target cells, thereby reducing the complexity of the mixture subjected to the selective cell engineering. In some embodiments, the apheresis may comprise leukapheresis, such as lymphapheresis. Such embodiments could separate white blood cells (e.g., lymphocytes), which are of particular interest to therapeutic and non-therapeutic uses of the present disclosure.

In some embodiments, the system may comprise (e.g. before the cell engineering module and, if present, before the apheresis module) an extraction module for drawing blood from a patient. In some embodiments, the system may comprise (e.g. after the cell selection and engineering module) a reinsertion module for transferring the modified target cells back into the patient.

In some embodiments, the system may comprise a vein-to-vein system. Such a vein-to-vein system may in particular further comprise: before the cell engineering module and, if present, before the apheresis module, the extraction module for drawing blood from a patient; and, after the cell selection and engineering module, the reinsertion module for transferring the modified target cells back into the patient. In some embodiments, the system may perform the cell engineering in a closed loop wherein the blood remains within the system from the extraction to the reinsertion. Maintaining a closed system can reduce contamination risks and reduce the quality control steps related thereto that are performed. A closed-loop vein-to-vein can further reduce the contamination risk and the quality control steps.

In some embodiments, the system may be a system for non-therapeutic applications (herein further referred to as a ‘lab system’). Compared to a vein-to-vein system, the lab system may have additional degrees of freedom in its design. For example, the lab system may not provide for reinsertion into a patient. The mixture may come from non-patient source(s) and can be any useful biological mixture, including artificial mixtures (e.g. cells in a buffer). The target cells can generally be any cells that are to be modified.

In some embodiments, the system may further comprise a cell sorting module (e.g. before and/or after the cell engineering module). In some embodiments, the system may further comprise a cell purification module (e.g. after the cell engineering module). Cell sorting and purification modules can allow the (modified) target cells to be separated from other components in the mixture (e.g. from contaminants, non-target cells, and/or non-modified target cells).

In some embodiments, the system may further comprise a flow generation unit (e.g. a pump) for inducing a flow of the mixture through the system (e.g. through the cell monitoring zone/component and engineering component).

In some embodiments, the system may be usable for cell therapy. In addition to non-therapeutic uses, the subject matter of the present disclosure can be beneficial in cell therapy, where it can address several of the issues currently faced in this field (cf. background).

In some embodiments, any feature of any embodiment of the fourth aspect may independently be as correspondingly described for any embodiment of any of the other aspects.

In a fifth aspect, the present disclosure relates to a method for cell engineering target cells in a mixture comprising the target cells and non-target cells, comprising: a) classifying individual target cells in the mixture, wherein classifying a cell comprises determining whether the cell is a target cell; b) tracking at least some of the target cells; and c) selectively modifying at least some of the tracked target cells, wherein the target cells and the non-target cells are randomly distributed.

In some embodiments, step b—and optionally step a—may comprise imaging of the individual target cells, for example computational imaging, for example lens-free imaging. (Computational microscopy) lens-free imaging faciliates high-throughput (supporting relatively high flow rate) cell monitoring simultaneously over a wide area (large field of view), thereby facilitating efficient classification and tracking of the individual cells over the full cell monitoring zone using a single imaging unit (as opposed to e.g. stitching together information/images from multiple imaging units). This can enable label-free cell monitoring, thereby addressing some or all of the problems associated with cell labelling (cf. Background).

In some embodiments, selectively modifying at least some of the tracked target cells may comprise selectively inserting and/or modifying a nucleic acid in the tracked target cells. The modification of the cells may include a genetic transformation.

In some embodiments, a composition of the mixture may be maintained throughout steps a, b, and up to c. In step c, at least some reagents may be added to the mixture. In some embodiments, nothing may be removed from the mixture throughout steps a, b, and c. In such latter embodiments, something may be added to the mixture (e.g. reagent, solvent, etc.) during and/or throughout steps a, b, and/or c. The selective modification can thus be based not on a separate cell sorting step (e.g. separating the target cells from the non-target cells), but on individually selecting for modification only those cells classified as target cells and tracked to be at one of the engineering sites.

In some embodiments, any feature of any embodiment of the fifth aspect may independently be as correspondingly described for any embodiment of any of the other aspects.

The subject matter of the present disclosure will now be described by a detailed description of several example embodiments thereof. Other embodiments of the subject matter disclosed herein can be configured according to the knowledge of the person skilled in the art without departing from the technical teachings of the present disclosure.

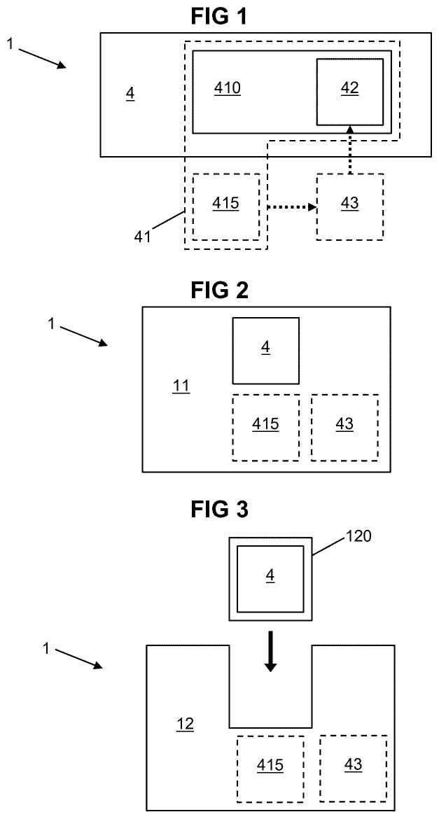

System for cell engineering target cells in a mixture FIG. 1 schematically depicts a system (1). At the core of this system (1) is a cell engineering module (4), which comprises a cell monitoring zone (410) and-within the cell monitoring zone (410) an engineering component (42). The cell monitoring zone (410) allows individual cells therewithin to be tracked and classified (including determining whether the cell is a target cell); while the engineering component (42) can selectively modify individual cells at one or more engineering sites (420). To this end, the engineering component (42) can be controlled so as to modify only selected individual cells.

In the operation of system (1), tracking and classifying of the individual cells may be performed using an imaging unit (415), such as a computational microscopy module. The cell monitoring zone (410) and the imaging unit (415) together form a cell monitoring component (41).

An operational system (1) may further comprise a control component (43) for controlling the engineering component (42). For instance, the control component (43) may signal/instruct the engineering component (42) when to perform cell poration and/or when to release a (bio)chemical (e.g. a nucleic acid to be introduced in a cell), etc. The control component (43) therefor receives information from the imaging unit (415). Depending on the implementation, this could be processed information, such as tracking information on one or more target cells, or information that is to be further processed by the control component (43) (e.g. raw imaging data that is to be further processed to track and classify individual cells therein). Based on this received information, the control component (43) then ideally controls the engineering component (42) such that only target cells (i.e. determined to be such during the classification) which are tracked to be at one of the engineering sites are modified.

Depending on the implementation, the above modules and components may be organized with respect to the system (1) in different ways. For example, the system (1) may be a device (11) as schematically depicted in FIG. 2, in which the cell engineering module (4)—and optionally the imaging unit (415) and control component (43)—is (semi-)permanently incorporated (e.g. in principle removable-e.g. in case of repair-but not intended to be removed in normal use).

Alternatively, the cell engineering module (4) may be formulated as a cartridge (120), and the system (1) may be comprised of the cartridge (120) and a device (12) for receiving the cartridge (120); e.g. as schematically depicted in FIG. 3. In this case, the imaging unit (415) and control component (43) may still be (semi-)permanently incorporated in the device (12), or they may likewise be formulated as parts which can be easily slotted in an out (not depicted in FIG. 3). Regardless, where the cartridge (120) does not include the imaging unit (415), the cell monitoring zone (not visualized in FIG. 3)—and the cartridge (120)—may generally be adapted for nevertheless allowing the imaging unit (415) to image the cell monitoring zone (410). To this end, the cell monitoring zone (410)—and the cartridge (120)—may for instance comprise at least one transparent surface (e.g. substantially transparent to the wavelengths used for the cell imaging by the imaging unit (415)).

### Cell Engineering Module

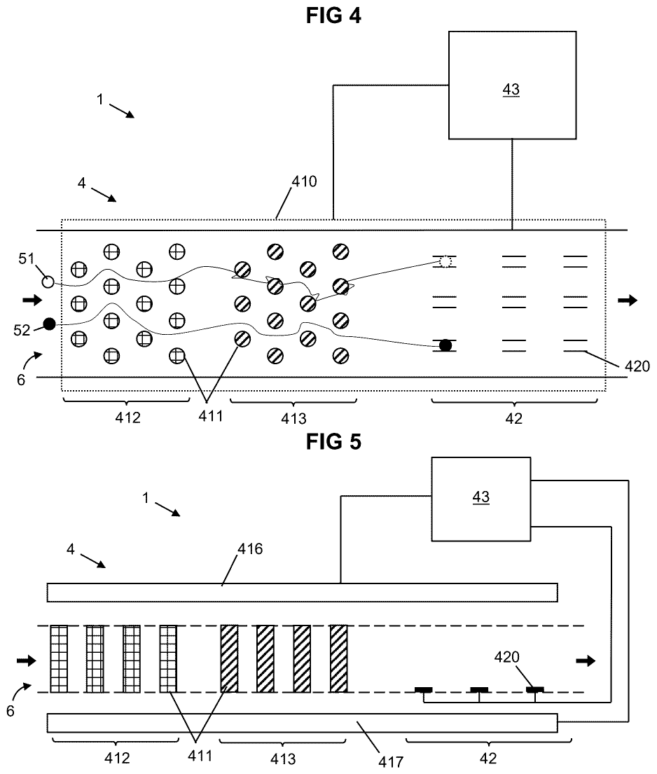

While a cell engineering module in accordance with the present disclosure could be realized in various ways, two exemplary approaches are described with reference to FIG. 4-FIG 7.

### Selective Cell Poration

In a first exemplary approach, depicted in FIG. 4 (top view) and FIG. 5 (side view), the system (1)—more specifically the cell engineering module (4)—comprises a fluidic channel (6) through which the mixture comprising the target cells (51) and non-target cells (52) flows (left to right in FIG. 4 and FIG. 5). A cell monitoring zone (410) is present within (part of) this fluidic channel (6), wherein individual target cells (51) can be tracked and classified.

This tracking and classifying can be done using an imaging unit-such as a computational microscopy module, for example a lens-free imaging unit—, which forms with the cell monitoring zone (410) a cell monitoring component (cf. supra). Depicted in FIG. 5 is a lens-free imaging unit comprising an illumination source (416) and a digital image sensor (417). Moreover, the top wall and bottom wall of the fluidic channel (6) are optically transparent, such that light from the illumination source (416) can propagate into the cell monitoring zone (410) and to the digital image sensor (417). In operation, the illumination source (416) lights up the objects—e.g. cells—in the cell monitoring zone (410). Interaction of the light from the illumination source (416) with light that reflects off the objects thereby results in an interference pattern, which is captured by the digital image sensor (417) as a digital hologram. This captured interference pattern can be used as is (i.e. individual objects/cells can be tracked directly in the captured interference pattern), or can be computationally processed into a detailed image (e.g. including depth information) of the objects/cells. Either way, interpretation (e.g. tracking and/or classification) of the captured interference pattern and/or constructed image may be done by the imaging unit as such, or by the control component (43) (cf. infra).

As depicted in FIG. 4 and FIG. 5, the mixture flows in a front part (i.e. upstream) of the cell monitoring zone (410) along/across a cell movement modifying surface (411). More specifically, as depicted, the fluidic channel (6) comprises two subzones (412, 413), each outfitted with structures-namely an array of pillars-having different/distinct cell movement modifying surfaces (411). Depending on the nature of the cell, different cells may interact (e.g. temporarily bind) differently (e.g. showing a substantial difference in the number of bind-and-release events, the average duration of a bind-and-release, etc.) with the movement modifying surface(s) (411); thereby influencing their movement through the cell monitoring zone (410). By tracking the movement—e.g. the speed, direction and/or path and/or interaction times-of individual cells (e.g. using the imaging unit), information can be gained on the nature of the individual cells, which—optionally in combination with their shape and/or size (which can also be obtained using the imaging unit) allows to classify the cells and determined whether the cell is a target cell (51). To that end, the one or more cell movement modifying surfaces (411) may be selected in function of the target cells (51) and non-target cells (52) which are expected to be present in the mixture, such that the influence on the movement of target cells (51) can be distinguished (e.g. in at least one of the subzones) from that of non-target cells (52). For instance, two individual cells may each follow a movement path-through the cell monitoring zone (410) to the engineering component (42)—as illustrated in FIG. 4, in which both cells interact differently—for example in the second subzone-with the pillars having the movement modifying surface (411). Accordingly, by tracking them individually, it can be determined that one can be classified as a target cell (51) while the other is a non-target or non-classified cell (52).

Note that, while the classification of the individual cells is described above as being based on cell movement and cell movement modifying surfaces (411), alternatives are possible. Indeed, depending on the target cells (51) and non-target cells (52) involved, their shape, size, transparency, scattering and/or speed as such (i.e. without using a cell movement modifying surface (411))—which can e.g. be obtained directly using the imaging unit—can already be sufficiently distinct to distinguish them.

Further downstream the cell monitoring zone (410), the mixture eventually flows through an engineering component (42). The engineering component (42) comprises engineering sites (420) at which individual cells can be selectively (e.g. under control by the control component (43), cf. infra) modified. Depending on the implementation, this may for instance involve (cf. supra) selectively opening the individual cell and/or triggering the release of one or more (bio)chemical for the modification. As depicted in FIG. 4 and FIG. 5, the engineering sites (420) are electrode pairs at which the selected cells can be electroporated. Reagents (e.g. (bio)chemicals) for the cell modification may be provided through one or more side channels (not visualized in FIG. 4 or FIG. 5; e.g. provided out of plane in FIG. 4) fluidically coupled to the engineering component (42). Depending on the application at hand, these side channels can be one or more central side channels which provide the reagents to all (or at least a collection of) engineering sites (420), or a side channel per engineering site (420) for provide the reagents in a precise (e.g. selectively controlled) and local manner to each engineering site (420).

A control component (43) is moreover present which receives information from the cell monitoring component (41) (e.g. from the imaging unit) and—based on this information—controls the engineering component (42). The information received from the cell monitoring component (41) could be fully processed cell tracking and classification data, partially processed or unprocessed (e.g. the raw interference patterns or constructed images). For instance, the classification of individual cells may be done by the cell monitoring component (41) as such (e.g. by the imaging unit) or by the control component (43).

Accordingly, after classification (cf. supra), at least the cells determined to be target cells (51)—and optionally all cells—are tracked in the cell monitoring zone (410) until they arrive at one of the engineering sites (420). Once a target cell (51) is tracked to be at an engineering site (420), the engineering component (42) is activated by the control component (43) to selectively modify the target cell (51). For instance, in FIG. 4, the electrode pair is triggered to electroporate the target cell (51) (indicated by the dotted line around the cell), thereby allowing the reagents for modifying the cell (e.g. by transfection) to enter the porated target cell (51). Conversely, non-target cells (52) do not trigger activation of the engineering component (42) and are left intact, so that reagents for modifying the cell cannot enter them and they thus do not undergo modification.

### Selective Administration of Reagent

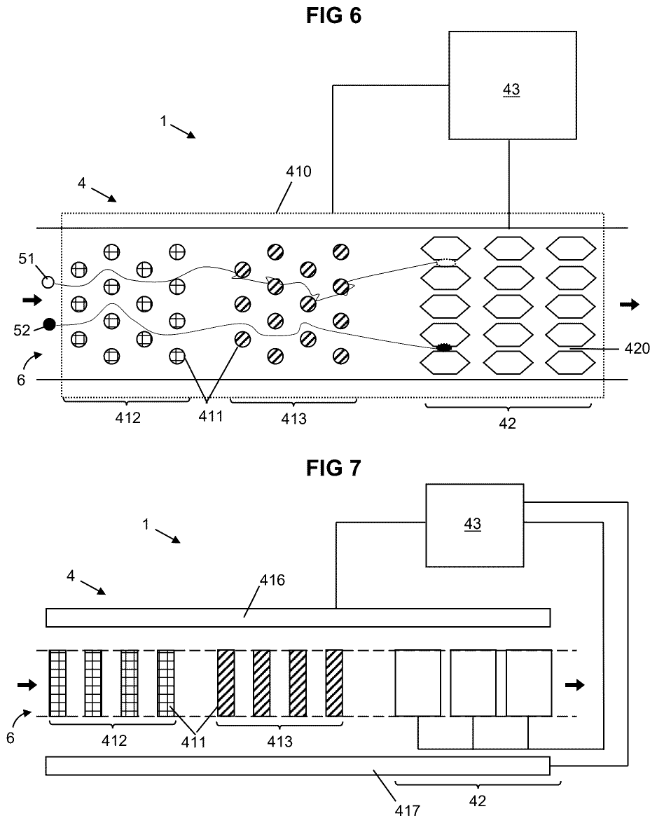

A second exemplary approach is depicted in FIG. 6 (top view) and FIG. 7 (side view) is substantially the same as the first approach, but—instead of electrode pairs for electroporation—the engineering sites (420) are narrow chokes at which all cells are porated through cell squeezing. Since in this approach all cells undergo poration, the selectivity of the modification is instead realized by selectively controlling—through the control module—the administration (e.g. release) of the reagents for modifying the cell such that it is available only locally at the engineering sites (420) where target cell (51) has been tracked to be. Accordingly, the one or more side channel (not visualized) for providing the reagents (e.g. (bio)chemicals) for the cell modification, may in this case comprise individual channel per engineering site.

### Selective Administration of Reagent

While not depicted separately, in an example implementation the selective cell poration of the first approach may be combined with the selective administration of reagent of the second approach. Accordingly, in this case the selectivity in ensured through both selectively opening the target cells and selectively locally administering reagents. For instance, the control component (43) may activate the engineering component (42) to trigger both the poration of a target cell (51) tracked to be at an engineering site (420) and the local release of one or more reagents at the engineering site (420). This dual level of selectivity further ensures that only the selected cells undergo the desired modification.

### Vein-to Vein System

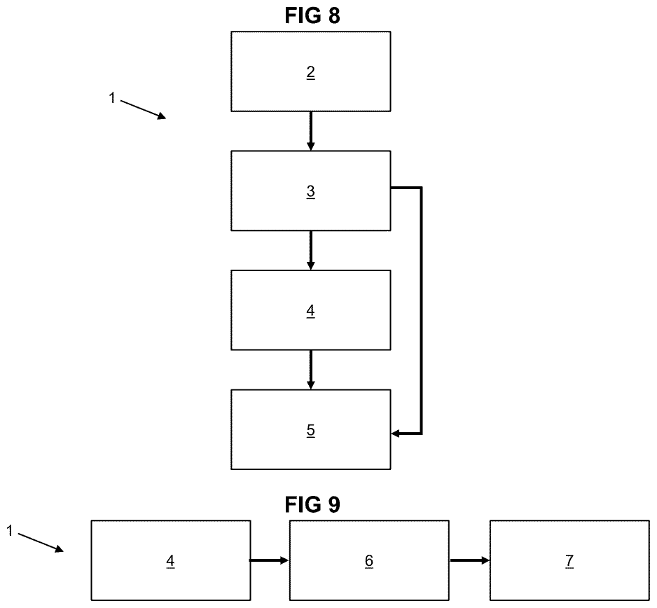

FIG. 8 schematically depicts a vein-to-vein system in accordance with embodiments of the present disclosure. A vein-to-vein system is a particular example of a system (1) in accordance with the present disclosure (which is in general not limited to vein-to-vein), in which the mixture comprising the target cells and non-target cells is blood extracted (for example from a vein) from a patient and the modified target cells are later reintroduced into a patient (e.g. the same patient). For example, this may be performed in a closed loop wherein the extracted blood remains in the system (1) from extraction to reinsertion. Moreover, the interaction of the extracted blood with any foreign (i.e. not native to the patient) products (e.g. reagents) and surfaces (e.g. in the system (1)) may be kept to a minimum. These ensure that the risk of contamination of the extracted blood is kept low and thereby also relaxes the quality control that needs to be performed on the blood prior to reinsertion.

Such a vein-to-vein system (1) may start with an extraction module (2) for drawing blood from a patient.

From the extraction module (2), the extracted blood may go to an apheresis module (3) for separating it into a fraction of interest and a residual fraction. For example, where the target cells are white blood cells (e.g. T cells or NK cells), the apheresis module (3) may be a leukapheresis module which separates the fraction of white bloods cells from the residual blood.

The fraction of interest—which comprises the target cells, but nonetheless also non-target cells—then goes to the engineering module (4), where the target cells undergo cell engineering (cf. supra). After cell engineering, the fraction of interest goes to a reinsertion module (5).

The residual fraction is not usually provided to the engineering module (4), or at least is not subjected to the same cell modification in the engineering module (4) (e.g. it may enter the engineering module (4) but follow a trajectory separate from the fraction of interest). For instance, the residual fraction may bypass the cell engineering module (4) and be directly provided to the reinsertion module (5).

In the reinsertion module (5), the fraction of interest and the residual fraction are recombined and transferred back into the patient.

It will be clear that the above is but one implementation of a vein-to-vein system (1), and that the functionality may be added or deleted from the above modules and/or operations may be interchanged between modules. For example, in some embodiments the apheresis may not be needed and this module could be omitted. Moreover, the recombination of the fraction of interest and the residual fraction could be performed in a dedicated recombination module. A quality control module might also be added between the engineering module (4) and the reinsertion module (5), in which case the recombination could (but does not need to) take place in—or before—the quality control module. Alternatively, the quality control functionality could be integrated into the reinsertion module (5).

### Lab System

FIG. 8 schematically depicts one of many potential configurations for a lab system, e.g. to perform research or another (non-therapeutical) application. As depicted, the system (1) comprises an engineering module (4), a cell sorting module (6) and a cell purification module (7).

It is to be understood that although specific embodiments, constructions, configurations, and materials have been discussed herein in order to illustrate the scope of the subject matter of the present disclosure. It will be apparent to those skilled in the art that various changes or modifications in form and detail may be made without departing from the scope of the present disclosure.

1. A system for cell engineering target cells in a mixture comprising the target cells and non-target cells, the system comprising:
a cell monitoring component configured to facilitate tracking and classifying individual cells in the mixture, wherein classifying a cell comprises determining whether the cell is a target cell;
an engineering component configured to selectively modify individual cells at one or more engineering sites; and
a control component configured to operate the engineering to selectively modify only target cells tracked to be at one of the engineering sites.

2. The system of claim 1, wherein selectively modifying individual cells comprises selectively inserting or modifying a nucleic acid in the individual cells.

3. The system of claim 2, wherein selectively modifying the individual cells comprises selectively transfecting the individual cells.

4. The system of claim 3, wherein transfecting the individual cells comprises electroporating, photoporating, cell squeezing, magnetofecting, chemical transfecting, or biochemically transfecting the individual cells.

5. The system of claim 1, wherein a composition of the mixture is maintained between classifying the individual cells and selectively modifying the individual cells.

6. The system of claim 1, wherein the cell monitoring component comprises an imaging unit.

7. The system of claim 6, wherein the imaging unit is a lens-free imaging unit.

8. The system of claim 1, wherein the cell monitoring component comprises a fluidic channel that comprises a cell movement modifying surface.

9. The system of claim 8, wherein the fluidic channel comprises one or more structures having the cell movement modifying surface.

10. The system of claim 8, wherein the fluidic channel comprises multiple subzones having different cell movement modifying surfaces.

11. The system of claim 1, wherein determining whether a particular cell is a target cell comprises determining a shape, size, transparency, scattering, speed, direction or path of the particular cell.

12. The system of claim 1, wherein the target cells are stem cells or immune cells.

13. The system of claim 12, wherein the target cells are peripheral blood mononuclear cells.

14. The system of claim 12, wherein the target cells are T cells or NK cells.

15. A method for cell engineering target cells in a mixture that comprises the target cells and non-target cells, the method comprising:
classifying individual target cells in the mixture, wherein classifying a given cell comprises determining whether the given cell is a target cell;
tracking at least some of the target cells; and
selectively modifying at least some of the tracked target cells, wherein the target cells and the non-target cells are distributed within the mixture.

16. The method of claim 15, wherein at least one of classifying individual target cells and tracking at least some of the target cells comprises computational imaging of individual target cells.

17. The method of claim 15, wherein selectively modifying at least some of the tracked target cells comprises selectively inserting or modifying a nucleic acid in the tracked target cells.

18. The method of claim 15, wherein a composition of the mixture is maintained from classifying individual target cells through tracking at least some of the target cells to selectively modifying at least some of the tracked target cells.

19. The method of claim 15, wherein determining whether the given cell is a target cell comprises determining a shape, size, transparency, scattering, speed, direction or path of the given cell.

20. The method of claim 15, wherein selectively modifying the individual cells comprises selectively electroporating, photoporating, cell squeezing, magnetofecting, chemical transfecting, or biochemically transfecting the individual cells.
